(19) United States
(12) Patent Application Publication
(10) Pub. No.: US 2024/0124909 A1
(43) Pub. Date: Apr. 18, 2024

(54) COMPOSITION AND METHOD FOR PRODUCTION OF FRUCTOSE

(71) Applicant: INDUSTRY-ACADEMIC COOPERATION FOUNDATION GYEONGSANG NATIONAL UNIVERSITY, Jinju-si, Gyeongsangnam-do (KR)

(72) Inventors: Seon Won KIM, Sejong-si (KR); Seong Hee JEONG, Gimhae-si, Gyeongsangnam-do (KR); Ji Bin PARK, Geumjeong-gu, Busan (KR); Moon Hyuk KWON, Jinju-si, Gyeongsangnam-do (KR)

(21) Appl. No.: 18/277,380

(22) PCT Filed: Feb. 28, 2022
(86) PCT No.: PCT/KR2022/002861

§ 371 Date: Aug. 16, 2023

(30) Foreign Application Priority Data
Mar. 30, 2021 (KR) 10-2021-0041008

(51) Int. Cl. C12P 19/18; C12N 9/10; C12P 19/02

(52) U.S. Cl. CPC C12P 19/18; C12N 9/1051; C12P 19/02; C12Y 204/01007

(57) ABSTRACT

Provided is a composition and a fructose production method, wherein the composition contains: sucrose phosphorylase or a microorganism expressing same; or a culture or lysate of the microorganism whereby fructose can be produced at low cost with high yield.

### TECHNICAL FIELD

The present invention relates to a composition and a method for production of fructose.

### BACKGROUND

Fructose is one of the main sugars found in fruits and is one of the major monosaccharides along with glucose. Produced primarily from sugar cane, sugar beets, and corn, it is the strongest of the sugars, with a sweetness of 1.2-1.8 times that of sucrose (table sugar), and is widely used as a commercial sweetener. Fructose is industrially produced by glucose isomerase from starch or by invertase from sucrose. However, the residual glucose produced with glucose or used as a substrate makes it difficult to achieve high-purity isolation of fructose due to the similar properties of hexose, which increases the cost of the production process.

The present invention utilizes a reaction to produce fructose from sucrose via sucrose phosphorylase (SPase) in order to facilitate the high-purity isolation of fructose while reducing the cost of the fructose production process. Sucrose used as a substrate with an additional substrate glycerol produces additional glucosylglycerol along with fructose through a conversion reaction. Glucosylglycerol is a sugar complex formed by glycosidic bonds between glucose and glycerol, and it is known to improve skin moisturization through its excellent water retention function, and to block water loss by penetrating the stratum corneum of the skin and improve skin elasticity, and is currently used as a highly functional ingredient in basic cosmetics for anti-aging and moisturizing by various global cosmetics companies. In addition, it has a sweetness of 55% of sucrose, but does not cause tooth decay, and can be used as a low-calorie sweetener because only 19% of the intake is utilized in the intestine, and it is also known to have a function as a prebiotic. It is easy to separate fructose from the mixture with glucosylglycerol, and since glucosylglycerol is an expensive functional substance, it is possible to obtain additional added value and reduce the production cost of fructose.

A sequential functional sugar production process can be applied using the produced fructose as a substrate without separating the fructose. The rare sugar psicose, which exists in very small amounts in nature, has an epimerized form of the third carbon of fructose and has a sweetness of 70% of sucrose, but since most of it is discharged when ingested, it has near-zero calories, which is only 5-10% of sucrose, and is attracting attention as a sucrose substitute sweetener. It also reduces abdominal fat by inhibiting the activity of abdominal lipogenic enzymes, and can be used as a sweetener for weight control or as an alternative sweetener for diabetics because it inhibits the absorption of glucose in the intestine, protects the islet beta cells of the pancreas, and improves insulin sensitivity. The enzymatic conversion method developed by Izumori enables the conversion of fructose to psicose using D-Psicose 3-epimerase (DPE). A sequential functional sugar production process was applied to produce glucosylglycerol and psicose, converting the fructose produced to psicose.

In addition, a process was applied to convert the fructose remaining after the psicose conversion reaction into mannitol to create additional value-added of fructose and to separate sucrose. Psicose 3-epimerase (DPE), which is currently used to produce psicose from fructose as a substrate, has a reaction equilibrium of around 30%, resulting in a large amount of fructose remaining after the reaction. The similar properties of fructose and psicose make it difficult to isolate psicose. Mannitol, a sugar alcohol, has a hydrogenated form of the second carbon of fructose and is used as an alternative sweetener for diabetics because it has a sweetness similar to sucrose, but with a calorie content of 1.6 kcal/g, which is lower than sucrose, and does not raise blood sugar levels. In addition, it does not cause tooth decay, has a strong cooling effect and is used to give food a minty aroma and flavor, and has low hygroscopicity, so it is also used as a coating for chewing gum, candy, and dried fruits. It is also used medicinally to lower intraocular and intracranial pressure and as an osmotic diuretic. Mannitol can be produced by the enzyme mannitol 2-dehydrogenase (MDH) using fructose as a substrate. In addition, formate dehydrogenase (FDH), which reduces NAD+ through the oxidation of formic acid, is introduced for smooth regeneration of NADH, which is used by mannitol dehydrogenase as a cofactor. By converting a large amount of fructose remaining after the psicose conversion reaction into another functional sugar alcohol, mannitol, the separation of psicose is facilitated and the added value of fructose is increased.

By applying the process to produce functional sugars sequentially, glucosylglycerol, psicose, and mannitol are ultimately produced. To date, the process of sequentially producing two or more functional sugars has not been studied before. The present invention was conceived to increase the commercial availability of high-value functional materials by developing a low-cost, high-efficiency, sequential functional sugar production process that maximizes the added value of the substrate.

### DETAILED DESCRIPTION OF THE INVENTION

### Technical Problem

It is an object of the present invention to provide a composition and a method for production of fructose with good production yields.

### Technical Solution

1. A composition for production of fructose comprising a sucrose phosphorylase or a microorganism expressing the same; or a culture or lysate of the microorganism.

2. The composition according to the above 1, wherein the sucrose phosphorylase comprises a sequence of any one of SEQ ID NO: 1 to 4, or a sequence having at least 80% sequence homology thereto.

3. The composition according to the above 1, wherein the microorganism endogenously or exogenously expresses the sucrose phosphorylase.

4. The composition according to the above 1, wherein the microorganism is a microorganism of the genus Escherichia or the genus Corynebacterium.

5. The composition according to the above 1, wherein the microorganism is induced to have dormant cells.

6. A composition for production of sugar comprising the composition according to the above 1; and an enzyme of a sugar production pathway utilizing fructose as a substrate, a microorganism expressing the enzyme, and a culture or lysate of the microorganism.

7. The composition according to the above 6, wherein the sugar production pathway utilizing fructose as a substrate is a psicose production pathway, a mannitol production pathway, a tagatose production pathway, or a sorbitol production pathway.

8. The composition according to the above 6, wherein the microorganism expressing sucrose phosphorylase further expresses an enzyme of a sugar production pathway utilizing fructose as a substrate.

9. A method for production of fructose, comprising reacting sucrose and glycerol with the composition of any one of the above 1 to 5.

10. The method according to the above 9, further comprising separating fructose from product.

11. The method according to the above 9, wherein the composition comprises the microorganism, and wherein the reaction is carried out in the microorganism.

12. The method according to the above 11, further comprising inducing the microorganism to have dormant cells by culturing it in a growth medium prior to the reaction of sucrose with sucrose phosphorylase.

13. The method according to the above 9, wherein the reaction is carried out in a reaction solution without a buffer.

14. A method for production of sugar comprising reacting sucrose and glycerol with the composition of any one of the above 6 to 8.

15. The method according to the above 14, wherein the sugar is sucrose, mannitol, tagatose or sorbitol, and wherein the composition comprises an enzyme of the sugar synthesis pathway, a microorganism expressing the same, or a culture or lysate of the microorganism.

### Effect of the Invention

The present invention can produce fructose with a high yield.

The present invention can reduce the cost for production of fructose.

### BRIEF DESCRIPTION OF THE DRAWINGS

FIG. 1 illustrates the production of fructose or downstream sugars from sucrose via an enzymatic reaction.

FIG. 2 shows the results of constructing a fructose production process using recombinant E. coli and comparing the activity of the enzymes. FIG. 2A shows fructose production by sucrose phosphorylase, FIG. 2B shows glucosylglycerol production, and FIGS. 2C and 2D show sucrose and glycerol consumption. Sucrose phosphorylase from Aloscardovia chrysettii is labeled Ac, sucrose phosphorylase from Bifidobacterium adolescentis is labeled Ba, sucrose phosphorylase from Bifidobacterium pseudorongum is labeled Bp, and sucrose phosphorylase from Thermomacrothrix daxensis is labeled Td.

FIG. 3 shows a comparison of the productivity of sucrose phosphatases with and without buffer. FIG. 3A shows the fructose productivity with and without buffer, and FIG. 3B shows the pH change with and without buffer. The sucrose phosphatase from Aloscardovia chrysettii is labeled Ac, the sucrose phosphatase from Bifidobacterium adolescentis is labeled Ba, the sucrose phosphatase from Bifidobacterium pseudorongum is labeled Bp, and the sucrose phosphatase from Thermomacrothrix daxensis is labeled Td. 0 indicates with buffer, and X indicates without buffer.

FIG. 4 is a graphical representation of the results of a comparison of sucrose phosphorylase activities using E. coli as a whole cell. FIG. 4A shows fructose productivity by sucrose phosphatase, FIG. 4B shows glucosylglycerol productivity by sucrose phosphatase, FIG. 4C shows sucrose consumption by sucrose phosphatase, and FIG. 4D shows glycerol consumption by sucrose phosphatase. Sucrose phosphorylase from Aloscardovia chrysettii is labeled Ac, sucrose phosphorylase from Bifidobacterium adolescentis is labeled Ba, sucrose phosphorylase from Bifidobacterium pseudorongum is labeled Bp, and sucrose phosphorylase from Thermanarothrix daxensis is labeled Td.

FIG. 5 is a high-performance liquid chromatography chromatogram of substances used or produced in the sequential functional sugar production process.

FIG. 6 is a diagrammatic representation of the results of the production of functional sugars using a sugar refinery. The first process shows the results of the fructose and glucosylglycerol production process, the second process shows the results of the sucrose production process, and the third process shows the results of the mannitol production process.

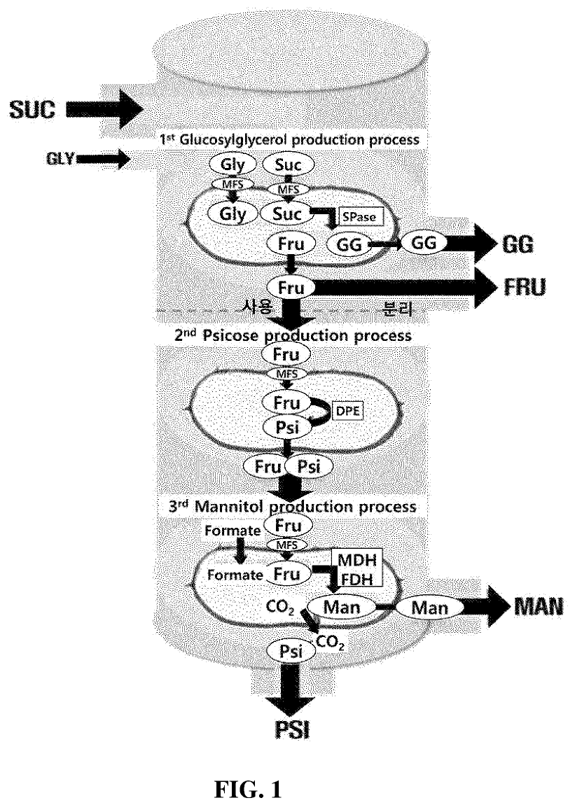

### FORM FOR PRACTICING THE INVENTION

The present invention will now be described in detail.

The present invention relates to compositions for production of fructose.

In an embodiment of the invention, the composition comprises a sucrose phosphorylase or a microorganism expressing the same; or a culture or lysate of the microorganism.

Sucrose phosphorylase (SPase) is an enzyme that uses sucrose as a substrate and phosphorylates it.

Specifically, sucrose phosphorylase can use sucrose and glycerol as substrates together to produce fructose and glucosylglycerol.

Since glycerol and glucosylglycerol have different physical properties, such as solubility, than fructose, fructose can be easily isolated from the mixture in high purity.

In an embodiment of the invention, as a sucrose kinase, those known as sucrose kinases and other proteins that can perform the same function in consideration of sequence homology can be used without limitation, for example, any one of SEQ ID NOs: 1 to 4 or a peptide consisting of a sequence having 80% or more sequence homology thereto may be used.

In an embodiment of the invention, the microorganism expressing sucrose phosphorylase may be endogenously or exogenously expressing said peptide.

In an embodiment of the invention, in the case of exogenously expressing said peptide, said microorganism may be introduced with a gene encoding said peptide.

In an embodiment of the invention, the introduction of the gene may be accomplished by using, without limitation, plasmids, viral vectors such as retroviruses, adenoviruses, and the like, or non-viral vectors known in the art.

In an embodiment of the invention, the microorganism may be prokaryotic or eukaryotic cell that can be cultured in a liquid medium and is capable of being cultured at the elevated temperatures described above. The microorganism may be, for example, bacteria, fungi, or combinations thereof. The bacteria may be gram-positive bacteria, gram-negative bacteria, or a combination thereof, and may be gram-positive bacteria in terms of increasing fructose productivity. The gram-negative bacteria may be of the genus Escherichia. The gram-positive bacteria may be of the genus Bacillus, the genus Corynebacterium, the genus Actinomyces, the genus Lactobacillus, or a combination thereof. Fungi can be yeasts, the genus Cluveromyces, or a combination thereof.

In an embodiment of the invention, the microorganism may be induced to have dormant cells.

In an embodiment of the invention, the induction to have dormant cells may be accomplished by culturing the microorganism to a stationary phase.

In an embodiment of the invention, cultivation to stationary phase may be performed in media with or without substrate, and cultivation in media containing substrate may be preferable in that it allows the microorganism to adapt to the substrate.

In an embodiment of the invention, culture to stationary phase may be performed by cultivation in a growth medium, which may include, without limitation, components known in the art for the culture of such microorganism.

As used herein, the term resting cell refers to a cultured cell in a state in which it is no longer proliferating. As used herein, the term stationary phase refers to a state in which a cell has passed the exponential phase in culture and has ceased to divide and proliferate, so that there is no increase in the cell population, and the synthesis and degradation of cellular components are in balance.

Therefore, a dormant cell according to the present invention means a cell in a state in which growth is completed and the expression of the above proteins in the cell is sufficient, such that when the microorganism is induced to have a dormant cell, the expression of sucrose phosphatase is maximized, which can maximize fructose production.

In an embodiment of the invention, the culture of microorganisms can include a medium containing the microorganisms after incubation, a medium from which the microorganisms were isolated after incubation, or a substance secreted by the microorganisms during incubation. The medium may be a solid medium or a liquid medium.

In an embodiment of the invention, the crude product of the microorganism may be a crude product of the microorganism by sonication or the like, and may comprise said protein in said microorganism.

The present invention also relates to a composition for production of sugar.

In an embodiment of the invention, the composition comprises said composition for production of fructose, and further comprises a substance necessary for production of sugar of a post-fructose pathway.

Fructose may be produced from sucrose by use of the composition for production of fructose, and the post-fructose pathway may be further progressed sequentially if the composition further comprises a substance necessary for production of the post-fructose pathway sugar.

In an embodiment of the invention, the substance required for production of sugar of the post-fructose pathway may be an enzyme of a sugar production pathway utilizing fructose as a substrate, microorganism expressing the enzyme, a culture or a crude preparation of the microorganism.

In an embodiment of the invention, the sugar to be produced may be any sugar that can be produced using fructose as a substrate, without limitation. For example, the sugar is a sugar in a sucrose production pathway, a mannitol production pathway, a tagatose production pathway, a sorbitol production pathway, and the like.

In an embodiment of the invention, an enzyme of the sugar production pathway may include all enzymes used from the use of fructose as a substrate to the production of the final target sugar. Various sugar production pathways are known in the art, as are the enzymes that participate in their production, and any known enzyme of such a known pathway may be used. The enzymes may be of various microbial origin.

In an embodiment of the invention, to give a specific example, if the sugar to be produced is sucrose, a sucrose epimerase may be used, for example, a sucrose epimerase from Clostridium hylemonae (GenBank ID: EEG74378.1, SEQ ID NO: 21). If the sugar to be produced is mannitol, mannitol dehydrogenase and formic acid dehydrogenase may be used additionally. For mannitol dehydrogenase, mannitol dehydrogenase from Lactobacillus reuteri (GenBank ID: WP 003669358, SEQ ID NO: 22) may be used, and for formic acid dehydrogenase, formic acid dehydrogenase from Mycobacterium vaccae (GenBank ID: BAB69476.1, SEQ ID NO: 23, MvFDH) may be used, but is not limited to.

In an embodiment of the invention, if there are additional substrates required for enzymes in the sugar production pathway that utilize fructose as a substrate, the composition for production of sugar of the present invention may further comprises such a substrate. For example, if the sugar is mannitol, sodium formate, a substrate for formate dehydrogenase, can be further used.

In an embodiment of the invention, in the composition for production of sugar of the present invention comprise the microorganism, a culture, or lysate of the microorganism, the microorganism may be included in addition to the microorganism expressing sucrose phosphorylase, or the microorganism expressing sucrose phosphorylase may further express enzymes of a sugar production pathway that utilizes fructose as a substrate.

In an embodiment of the invention, the microorganism expressing sucrose phosphorylase may be endogenously or exogenously expressing the peptide.

In an embodiment of the invention, in the case of exogenously expressing the peptide, the microorganism may be one into which a gene encoding the peptide has been introduced.

In an embodiment of the invention, the introduction of the gene may be accomplished by using, without limitation, plasmids, viral vectors such as retroviruses, adenoviruses, and the like, or non-viral vectors known in the art.

In an embodiment of the invention, the microorganism may be a prokaryotic or eukaryotic cell, capable of being cultured in a liquid medium, and capable of being cultured at the elevated temperatures described above. The microorganisms may be, for example, bacteria, fungi, or combinations thereof. The bacteria may be gram-positive bacteria, gram-negative bacteria, or a combination thereof, and may be gram-positive bacteria in terms of increasing glucosylglycerol productivity. The gram-negative bacteria may be of the genus Escherichia. The gram-positive bacteria may be of the genus Bacillus, the genus Corynebacterium, the genus Actinomyces, the genus Lactobacillus, or a combination thereof. Fungi may be yeasts, the genus Cluveromyces, or a combination thereof.

In an embodiment of the invention, the microorganism may be induced to have dormant cells.

The present invention also relates to a method for production of fructose.

In an embodiment of the invention, the method for production of fructose comprises reacting sucrose and glycerol with the composition for production of fructose.

In an embodiment of the invention, since the composition comprises sucrose phosphorylase or a microorganism expressing it; or a culture or lysate of the microorganism; the reaction may be carried out, for example, in a microorganism.

In an embodiment of the invention, the reaction may be carried out, for example, at a temperature of 30° C. to 90° C., but is not limited thereto. Within the above range, the reaction may be performed at a temperature of 30° C. to 90° C., 30° C. to 80° C., 30° C. to 70° C., 40° C. to 80° C., 40° C. to 70° C., 45° C. to 70° C., 50° C. to 70° C., and the like.

In an embodiment of the invention, the ratio of the substrate sucrose to glycerol in the reaction is not particularly limited, for example, the molar ratio may be 1:0.1 to 10. Within the above range, it may be 1:0.1 to 10, 1:0.1 to 8, 1:0.5 to 8, 1:1 to 8, 1:1 to 5, and the like.

In an embodiment of the invention, if the composition comprises the microorganism, its culture or lysate, the method of the invention may further comprise inducing the microorganism to have dormant cells, prior to the reaction. In such cases, the production of fructose can be maximized.

In an embodiment of the invention, the induction to have dormant cell may be performed by culturing the microorganism to a stationary phase.

In an embodiment of the invention, culture to stationary phase may be performed in media with or without substrate, and cultivation in media with substrate may be preferable in that it adapts the microorganism to the substrate.

In an embodiment of the invention, culture to stationary phase may be performed by cultivation in growth media, which may include, without limitation, components known in the art for cultivation of such microorganisms.

In an embodiment of the invention, for example, the reaction may be carried out in a reaction solution that does not contain buffer. In the production of the target product using an enzyme or a microorganism, a buffer is usually used to maintain the pH in a certain range, otherwise the enzyme activity is reduced and the production yield is significantly reduced. The method of the present invention exhibits excellent production yields even when the reaction is carried out in a reaction solution that does not contain a buffer, thereby reducing the time, cost, etc. required for production of fructose.

When sucrose and glycerol are reacted with the above composition for production of fructose, a mixture of fructose and glucosylglycerol can be obtained, and fructose can be easily separated from glycerol and glucosylglycerol due to the large difference in physical properties such as solubility from glycerol and glucosylglycerol. Therefore, the method of the present invention may further comprise separating fructose from the reaction mixture.

The present invention also relates to a method for production of sugar.

In an embodiment of the invention, the method for production of sugar of the present invention includes reacting sucrose and glycerol with the composition for production of sugar.

The composition for production of sugar comprises the composition for production of fructose, so that fructose may be produced by the reaction of sucrose and glycerol with the composition for production of fructose, and further comprises a substance necessary for production of sugar of the post-fructose pathway, so that a reaction for production of sugar of the post-fructose pathway may further proceed using fructose as a substrate.

In an embodiment of the invention, the reaction may be carried out, for example, in a microorganism.

In an embodiment of the invention, if the composition comprises the microorganisms, its cultures or lysate, the method of the invention may further comprise inducing the microorganism to have dormant cells, prior to the reaction. In such cases, the production of fructose can be maximized.

In an embodiment of the invention, the induction to have dormant cells may be performed by culturing the microorganism to a stationary phase.

In an embodiment of the invention, culture to stationary phase may be performed in media with or without substrate, and cultivation in media with substrate can be preferable in that it adapts the microorganism to the substrate.

In an embodiment of the invention, culture to stationary phase may be performed by cultivation in growth media, which may include, without limitation, component known in the art for cultivation of such microorganism.

In an embodiment of the invention, the reaction may be carried out in a reaction solution that does not contain buffer, for example. In the production of the target product using an enzyme or a microorganism, a buffer is usually used to maintain the pH in a certain range, otherwise the enzyme activity is reduced and the production yield is significantly reduced. The method of the present invention exhibits excellent production yields even when the reaction is carried out in a reaction solution that does not contain a buffer, thereby reducing the time, cost, etc. required for production of sugar.

The present invention will now be described in more detail with reference to the following embodiments.

### EXAMPLE

1. Establishment of Fructose Production Process Using Recombinant E. coli and Comparison of Enzyme Activities

Using the amino acid sequence of the previously used thermophilic and highly active Bifidobacterium adolescentis-derived sucrose phosphatase (GenBank ID: AA033821.1, SEQ ID NO:1, BaSPase) as a template, amino acid sequence results from various strains according to homology were obtained using NCBI's BLAST (https://blast.ncbi.nlm.nih.gov/Blast.cgi). Among them, Bifidobacterium adolescentis-derived sucrose phosphatase with 83.8% homology with Bifidobacterium pseudolongum-derived sucrose phosphatase (GenBank ID: WP_026643821.1, SEQ ID NO:2, BpSPase), 76.2% homology with Alloscardovia criceti-derived sucrose phosphatase (GenBank ID: WP_018142968. 1, SEQ ID NO:3, AcSPase) and a sucrose phosphatase from Thermanaerothrix daxensis (GenBank ID: WP_054521739.1, SEQ ID NO:4, TdSPase) with 55.1% homology are selected. Aloscardovia crisetti and Bifidobacterium pseudorongum were obtained from the Korean Collection for Type Cultures (KCTC) as KCTC 5819 and KCTC 3234, and Thermanaerothrix daxensis was obtained from the German Collection of Microorganisms and Cell Cultures GmbH (DSMZ) as DSM 23592.

The genomes of the distributed strains were purified and PCR was performed to include the respective sucrose phosphatase coding gene sequences. Primer pairs of SEQ ID NO:6 and 7 were used to PCR the sucrose phosphatase coding gene sequence from Bifidobacterium pseudorongum (SEQ ID NO:5), and primer pairs of SEQ ID NO:9 and 10 were used to PCR the sucrose phosphatase coding gene sequence from Aloscardovia chrysettii(SEQ ID NO:8), Primer pairs of SEQ ID NO:12 and 13 were used to perform PCR of the sucrose phosphatase coding gene sequence from Thermanarothrix daxcensis (SEQ ID NO:11). The primers were purchased from BIONEER (Korea), and the PCR was performed using TaKaRa (Japan) PCR Thermal Cycler Dice under the recommended conditions using Phusion DNA Polymerase from ThermoFisher SCIENTIFIC (USA) as the polymerase.

The obtained PCR products were introduced into the expression vector pTrc99A of E. coli containing the trc promoter using the restriction enzymes KpnI and XbaI from NEW ENGLAND BioLabs (NEB, UK), resulting in the recombinant vectors pT-AcSPase, pT-BpSPase and pT-TdSPase. The recombinant vectors were introduced into Escherichia coli BW 25113 Δglpk strain lacking glycerol phosphokinase (glpk) by a chemical method referring to Sambrook et al. Molecular Cloning 3rd (2001). The transformed recombinant E. coli was stored at −80° C. until use.

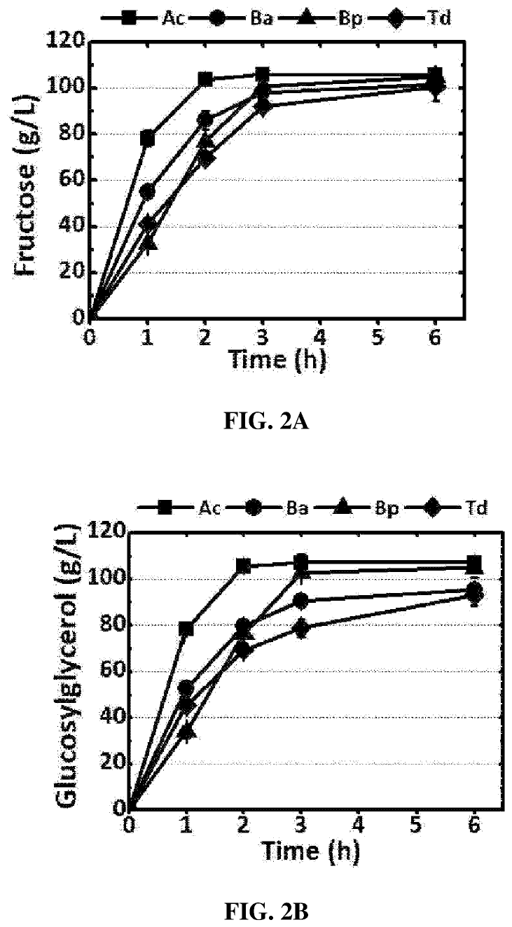

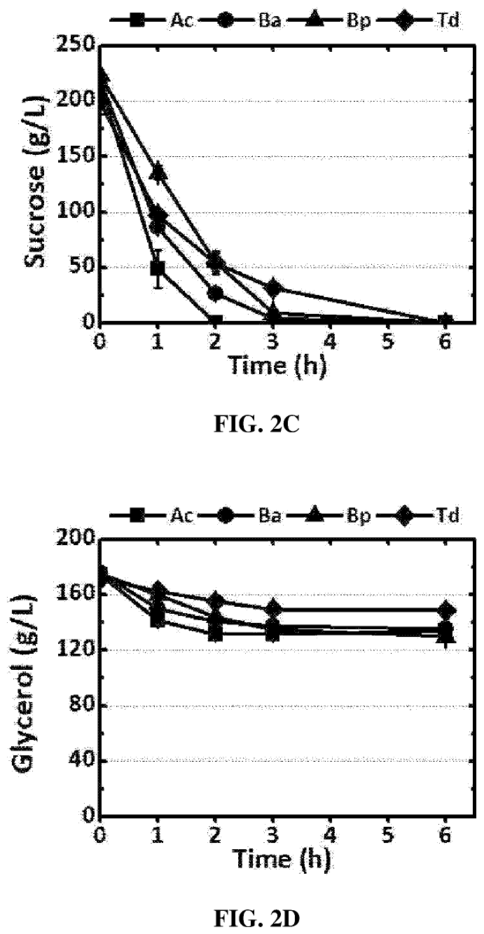

Recombinant E. coli was grown in large quantities in flasks and was obtained at a cell concentration of OD 40 and used as a biocatalyst for the whole-cell conversion reaction. 250 mM PIPES (pH 7.0) containing 200 g/L sucrose and 200 g/L glycerol was used as the conversion solution, and the conversion reaction was carried out for 6 hours at a conversion temperature of 60° C. with a stirring speed of 180 rpm to compare the activity of each enzyme. Sugars were analyzed using High Performance Liquid Chromatography (HPLC) by SHIMADZU (Japan), using a KR100-5NH2 (250×4.6 mm, 5 μm) column from Kromasil (Sweden) with 85% acetonitrile as mobile phase and a Reflective Index detector (RID). The results are shown in FIG. 2.

Referring to the fructose production results in FIG. 2A, the recombinant E. coli with sucrose phosphatase from Aloscardovia chrysettii not only had the fastest production rate, reaching reaction equilibrium in less than 2 hours, but also the highest yield, with a final fructose production of 105.8 g/L. The recombinant E. coli introducing Bifidobacterium pseudorongum-derived sucrose phosphatase and Thermanarothrix daxcensis-derived sucrose phosphatase had similar fructose productivity to the conventional Bifidobacterium adolescentis-derived sucrose phosphatase, and all reached reaction equilibrium within 3 hours.

Referring to the glucosylglycerol results in FIG. 2B, similar to the fructose production results, the recombinant E. coli introducing sucrose phosphatase from Aloscardovia chrysetti was the most productive, producing a final glucosylglycerol yield of 107.3 g/L.

The sucrose and glycerol consumption results in FIGS. 2C and 2D show that all of the 200 g/L sucrose was consumed, while the 200 g/L glycerol was consumed in the same amount as the 200 g/L sucrose molar ratio, with approximately 50 g/L consumed and 150 g/L remaining. Glycerol is not expected to have any effect on the high-purity separation of fructose, as its properties are distinctly different from fructose.

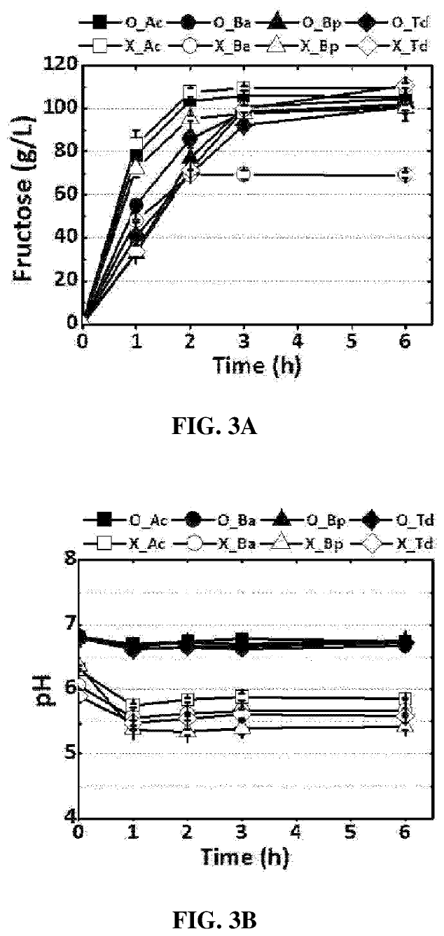

Every enzyme has its own optimal active pH, but the buffers used to maintain the pH are expensive, increasing the final production cost and increasing the impurities in the reaction solution, making it difficult to achieve high-purity separation of the product. These problems make it difficult to advance to industrial processes, so it is advantageous to find enzymes with low pH dependence. To apply this to the present invention, the pH-dependent activity of the newly discovered sucrose phosphatase was compared. In the above conversion reaction, 250 mM Pepes buffer was used to maintain pH 7.0, which is known to be the optimal active pH of sucrose phosphatase, and the productivity of sucrose phosphatase was compared using a reaction solution without buffer under the same conditions. The pH dependence of all sucrose phosphatases selected earlier was compared with and without Pepes buffer under the same strain and production conditions as in the previous experiment. The results are shown in FIG. 3.

Referring to FIG. 3A, which shows fructose productivity with and without buffer, the sucrose phosphatase from Bifidobacterium pseudorongum showed improved productivity in the absence of buffer compared to the presence of buffer. Sucrose phosphorylase from Aloscardovia chrysettii and Thermomacrothrix daxensis had similar productivity without buffer as with buffer. However, sucrose phosphatase from Bifidobacterium adolescentis showed the largest pH-dependent activity difference, with a 32% decrease in productivity without buffer compared to buffer.

Referring to FIG. 3B, which shows the change in pH with and without buffer, 250 mM PIPES (pH 7.0) was found to maintain a pH between 6.6-6.8 during the conversion reaction regardless of the origin of the sucrose phosphatase, whereas in the absence of buffer, the pH dropped to between 5.4-5.8 after 1 h of conversion reaction and remained there for the remainder of the conversion reaction. As a result, Bifidobacterium adolescentis-derived sucrose phosphatase showed a large decrease in activity upon pH change, suggesting that it cannot be used as an efficient production enzyme. However, the newly identified sucrose phosphatases from Aloscardovia chrysettii, Bifidobacterium pseudorongum, and Thermanarothrix daxensis did not appear to be pH-dependent, confirming their value as production enzymes for efficient and economical industrial processes in the future.

2. Establishment of a Fructose Production Process Using Recombinant Corynebacterium and Comparison of Enzyme Activities

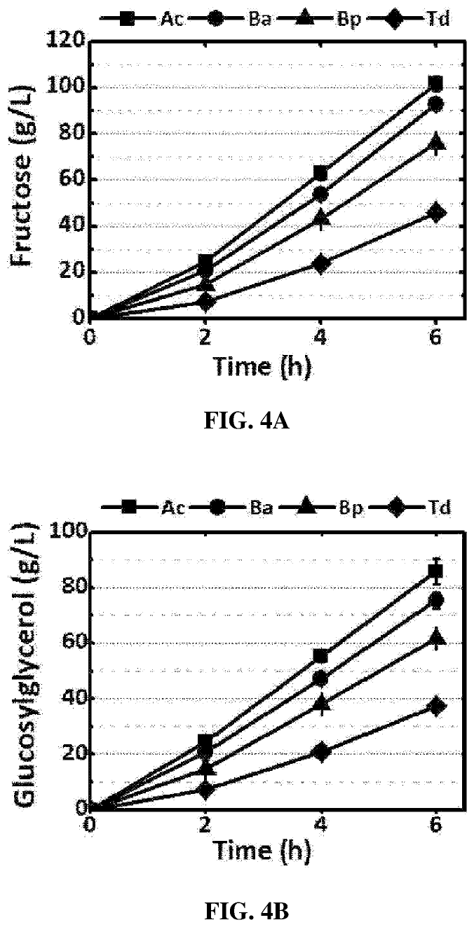

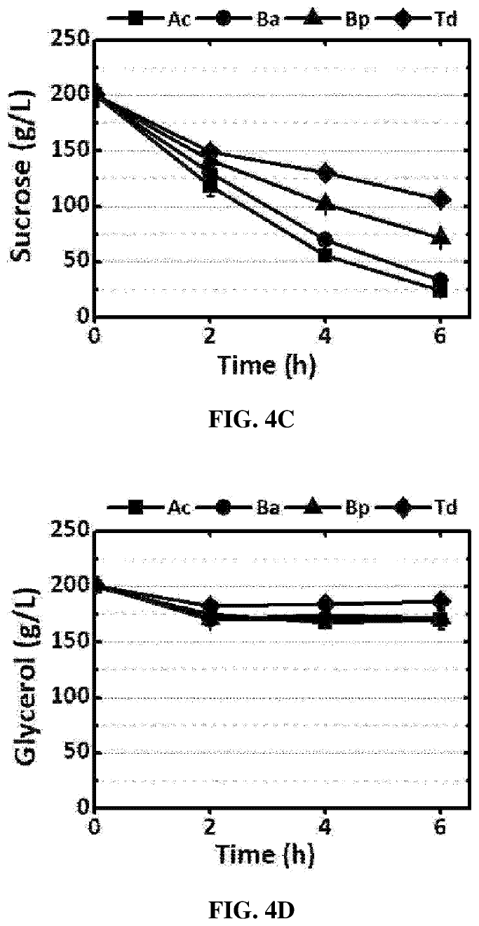

To establish a process favorable to high temperatures, we compared the activity of the new sucrose phosphorylase using Corynebacterium, a Gram-positive bacterium with a thick cell wall that is thermostable and safe as a GRAS strain. The genome of each purified strain was PCRed to include the sucrose phosphatase coding gene sequence as a template. To use BamHI and NotI as restriction enzymes, PCR was performed using primer pairs of SEQ ID NO:15 and 16 to include the sequence of SEQ ID NO:14 with substitutions for the BamHI and NotI positions in the sucrose phosphatase coding gene from Aloscardovia crisetti. PCR was performed using primer pairs of SEQ ID NO:17 and 18 to include the sucrose phosphatase coding gene from Bifidobacterium pseudorongum (SEQ ID NO:5), and primer pairs of SEQ ID NO: 19 and 20 to include the sucrose phosphatase coding gene from Thermophilus daxensis (SEQ ID NO: 11). The obtained PCR product was introduced into the E. coli-Corynebacterium shuttle vector pCES-H30 containing a strongly synthetic promoter H30 for Corynebacterium using the restriction enzymes BamHI and NotI (Yim S S, et al, 2013. Isolation of fully synthetic promoters for high-level gene expression in Corynebacterium glutamicum) to obtain the recombinant vectors pCES-H30-AcSPase, pCES-H30-BpSPase and pCES-H30-TdSPase. The recombinant vectors were introduced into wild-type Corynebacterium glutamicum ATCC 1302 by electroporation method referring to Eggeling et al. Handbook of Corynebacterium glutamicum (2005). The transformed recombinant Corynebacterium was stored at −80° C. until use. The recombinant wild-type Corynebacterium glutamicum ATCC 13032 strain introducing pCES-H30-AcSPase, pCES-H30-BaSPase, pCES-H30-BpSPase and pCES-H30-TdSPase was obtained at a cell concentration of OD 40 and used as a biocatalyst for the whole-cell conversion reaction, and the fructose production conversion reaction was carried out under the same conversion conditions as the whole-cell conversion reaction using E. coli. The results are shown in FIG. 4.

Referring to the fructose productivity results in FIG. 4A, same with the results in E. coli, the best productivity was achieved in Corynebacterium transfected with the Aloscardovia chrysetti-derived sucrose phosphatase, producing 101.6 g/L of fructose after a 6-hour conversion reaction, confirming that the rate of glucosylglycerol production in Corynebacterium was significantly reduced compared to that in E. coli.

Referring to the glucosylglycerol production results in FIG. 4B, similar to the fructose production results, the recombinant Corynebacterium introducing sucrose phosphatase from Aloscardovia chrysetti was the most productive, producing 85.9 g/L of glucosylglycerol after the final 6 hours of conversion reaction.

Referring to the sucrose and glycerol consumption results in FIG. 4C and FIG. 4D, it can be seen that sucrose and glycerol were consumed in inverse proportion to the fructose production of each sucrose phosphatase.

Although the use of Corynebacterium as a fructose production strain showed lower productivity compared to E. coli, increasing the expression of sucrose phosphatase in Corynebacterium will enable the application of a high-temperature fructose production process that maximizes the productivity and production cost efficiency of the fructose production process by establishing a strain that is similar in productivity to E. coli but more stable at high temperatures.

3. Development of a Sequential Production Process of Sucrose and Mannitol from Sucrose

After confirming that the process for producing fructose from sucrose was successfully established, we sequentially applied the process for producing sucrose and mannitol developed in the previous study.

Recombinant E. coli introducing sucrose phosphatase from Aloscardovia chrysettii, which showed the best productivity in the comparison of the above enzymes, was grown to a cell concentration of OD 40 and subjected to a 2 h whole-cell conversion reaction in 250 mM PIPES (pH 7.0) conversion solution containing 200 g/L sucrose and 200 g/L glycerol as substrates at a conversion temperature of 60° C. and a stirring speed of 180 rpm. The cells and reaction solution were then separated by centrifugation for 15 minutes at 3,500 rpm, and the separated reaction solution was used as the substrate and reaction solution for the second process, the psicose production process.

Recombinant Corynebacterium glutamicum transformed with a gene encoding a Clostridium hylemonae-derived cyclic epimerase (GenBank ID: EEG74378.1, SEQ ID NO:21, ChDPE) was obtained at a cell concentration of OD 40 and used as a biocatalyst, and 0.12 mM manganese, an active cofactor of the enzyme, is added. The whole-cell conversion reaction was carried out for 1 hour at a conversion temperature of 60° C. and a stirring speed of 180 rpm. After the reaction, the reaction solution separated by the same method was used as substrate and reaction solution for the third mannitol production process.

The gene encoding Lactobacillus reuteri-derived mannitol dehydrogenase (GenBank ID: WP_003669358, SEQ ID NO:22, LrMDH) and Mycobacterium vaccae-derived formic acid dehydrogenase (GenBank ID: BAB69476. 1, SEQ ID NO:23, MvFDH), recombinant Corynebacterium glutamicum introduced with the gene encoding MvFDH was obtained at a cell concentration of OD 40 and used as a biocatalyst, and sodium formate, used as a substrate for formic acid dehydrogenase, was added at a molar concentration equal to the expected residual fructose concentration. In addition, since the mannitol conversion reaction shows good activity at an initial pH of 6.0, the pH was corrected using 50% formic acid, and the conversion reaction was carried out for 12 hours at a conversion temperature of 45° C. and a stirring speed of 180 rpm.

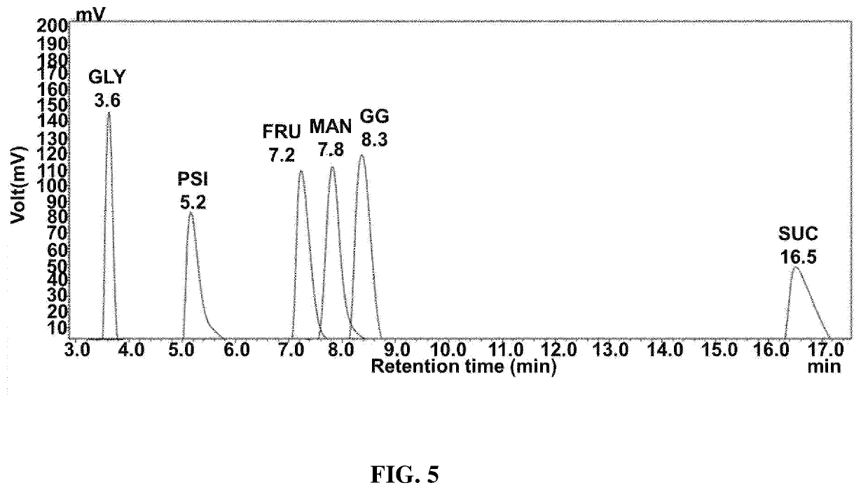

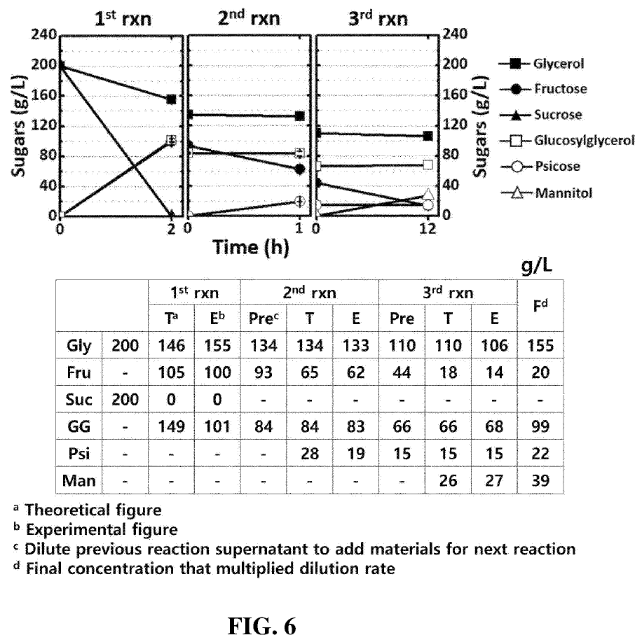

The conversion yield of each of the conversion reactions developed to date in this study is 100% relative to the concentration of sucrose for the fructose production process, 30% relative to fructose for the sucrose production process, and 60% relative to fructose for the mannitol production process. The high-performance liquid chromatography chromatograms of each substance are shown in FIG. 5, and the conversion reaction results are shown in FIG. 6.

Referring to FIG. 6, the first process, the fructose conversion reaction, produced 100 g/L of fructose according to the conversion yield using all sucrose, with 101 g/L of glucosylglycerol as a by-product of the reaction, and 155 g/L of glycerol remained. During the subsequent centrifugation process to separate the cells from the reaction solution, the cell membrane of the pre-cellular E. coli used as a whole cell became brittle due to prolonged exposure to heat, mixing with the reaction solution and diluting the sugar concentration, resulting in a fructose concentration of 93 g/L, a glucosylglycerol concentration of 84 g/L, and a glycerol concentration of 134 g/L. After the second conversion process, the psicose conversion reaction, 62 g/L of fructose remained, similar to the theoretical yield, suggesting that the same amount of fructose was used as in theory, but 19 g/L of psicose was produced, lower than the expected concentration of 28 g/L. The decreased yield of psicose is believed to be due to a decreased conversion rate as 93 g/L was used, which is lower than the 400 g/L used in the original sucrose production process. In the third conversion process, the mannitol conversion reaction, the initial sugar concentration was diluted to 44 g/L fructose, 66 g/L glucosylglycerol, 110 g/L glycerol, and 15 g/L sucrose with the addition of an additional substrate, formic acid, and pH correction. After the mannitol conversion reaction, 27 g/L of mannitol was produced, equal to the theoretical yield, 14 g/L of fructose remained, and glycerol, glucosylglycerol, and sucrose all remained in the same amount as before the reaction. Taken together, from 200 g/L sucrose and 200 g/L glycerol, 68 g/L, 15 g/L, and 27 g/L of the final three functional sugars, glucosylglycerol, psicose, and mannitol, respectively, were produced. However, since each reaction solution was diluted more by factors other than the addition of the required material, the yields were multiplied by a dilution factor to calculate the yield according to original reaction concentration. The dilution factor was calculated by comparing the amount of glycerol and glucosylglycerol after the first reaction to the amount remaining after the final reaction. Multiplying by the dilution factor, we found that the final glucosylglycerol, psicose, and mannitol yields were 99 g/L, 22 g/L, and 39 g/L.

This confirms that the concept of a sugar refinery that produces three functional sugars from sucrose in sequence through the application of a sequential functional sugar production process is viable and can be successfully implemented. This is a highly efficient sugar refinery that maximizes added value from inexpensive sucrose.

1. (canceled)

2. The composition according to claim 6, wherein the sucrose phosphorylase comprises a sequence of any one of SEQ ID NO: 1 to 4, or a sequence having at least 80% sequence homology thereto.

3. The composition according to claim 6, wherein the microorganism endogenously or exogenously expresses the sucrose phosphorylase.

4. The composition according to claim 6, wherein the microorganism is a microorganism of the genus Escherichia or the genus Corynebacterium.

5. The composition according to claim 6, wherein the microorganism is induced to have dormant cells.

6. A composition for production of sugar comprising:
a composition for production of fructose comprising a sucrose phosphorylase or a microorganism expressing the same; or a culture or lysate of the microorganism; and
an enzyme of a sugar production pathway utilizing the fructose as a substrate, a microorganism expressing the enzyme, or a culture or lysate of the microorganism.

7. The composition according to claim 6, wherein the sugar production pathway utilizing the fructose as Hall the substrate is a psicose production pathway, a mannitol production pathway, a tagatose production pathway, or a sorbitol production pathway.

8. The composition according to claim 6, wherein the microorganism expressing the sucrose phosphorylase further expresses the enzyme of Hall the sugar production pathway utilizing the fructose as Hall the substrate.

9. A method for production of fructose, comprising reacting sucrose and glycerol with a composition comprising a sucrose phosphorylase or a microorganism expressing the same, or a culture or lysate of the microorganism.

10. The method according to claim 9, further comprising separating the fructose from product.

11. The method according to claim 9, wherein the composition comprises the microorganism, and wherein the reaction is carried out in the microorganism.

12. The method according to claim 11, further comprising inducing the microorganism to have dormant cells by culturing the microorganism in a growth medium prior to the reaction of the sucrose with the sucrose phosphorylase.

13. The method according to claim 9, wherein the reaction is carried out in a reaction solution without a buffer.

14. A method for production of sugar comprising reacting sucrose and glycerol with a composition for production of fructose comprising a sucrose phosphorylase or a microorganism expressing the same, or a culture or lysate of the microorganism.

15. The method according to claim 14, wherein the sugar is sucrose, mannitol, tagatose or sorbitol, and wherein the composition further comprises an enzyme of Hall the sugar synthesis pathway, a microorganism expressing the same, or a culture or lysate of the microorganism.

16. The composition according to the claim 9, wherein the sucrose phosphorylase comprises a sequence of any one of SEQ ID NO: 1 to 4, or a sequence having at least 80% sequence homology thereto.

17. The composition according to claim 9, wherein the microorganism is a microorganism of the genus Escherichia or the genus Corynebacterium.

18. The composition according to the claim 14, wherein the sucrose phosphorylase comprises a sequence of any one of SEQ ID NO: 1 to 4, or a sequence having at least 80% sequence homology thereto.

19. The composition according to claim 14, wherein the microorganism is a microorganism of the genus Escherichia or the genus Corynebacterium.


SEQUENCE LISTING (23 sequences)

SEQ ID NO: 1 - protein, 504 aa, Bifidobacterium adolescentis [sequence omitted]

SEQ ID NO: 2 - protein, 505 aa, Bifidobacterium pseudolongum
MKNKVQLITYADRVGDGNLASLTDILRTRFAGVYEGVHILPFFTPFDGADAGFDPIDHTKVDPRLGDWDDIAELSKTHDIMVDAIVNHMSWQSRQFQDVLKHGEESEYYPMFLTMSSVFPDGATEEELAGIYRPRPGLPFTHYTFAGKTRLVWVTFTPQQVDIDTDSAEGWAYLMSIFDRMGTSHVKYIRLDAVGYGAKEAGTSCFMTPKTFALISRLREEGAKRGLEILIEVHSYYKKQVEIAAKVDRVYDFALPPLLLHSLFTGRVDALAHWTEIRPNNAVTVLDTHDGIGVIDIGSDQLDRSLKGLVPDEDVDAMVETIAKNTHGESKAATGAAASNLDLYQVNSTYYSALGGNDQHYIAARAVQFFLPGVPQVYYVGALAGSNDMELLKRTNVGRDINRHYYTTAEIDANLERPVVRALNALAKFRNELPAFDGGFNYAVDGETMSFTWNDGATSATLRFTPSRGMGADNAQPVAVLTWADAAGEHTSDDLIANPPVVHMD

SEQ ID NO: 3 - protein, 500 aa, Unknown
  Alloscardovia criceti
MKNKVQLITYADRLGDGTLQSMTETIRKHFDGVYEGVHILPFFTPFDGADAGFDPVDHTQVDPRLGSWDDVAELSKTHDIMVDTIVNHMSWESKQFQDVMAKGEESEYYPMFLTMSSIFPDGVTEEDLTAIYRPRPGLPFTHYTWGGKTRLVWTTFTPQQVDIDTDSEMGWNYLLTILDQLSQSHVSQIRLDAVGYGAKEKNSSCFMTPKTFKLIERIKAEGEKRGLETLIEVHSYYKKQIEIASKVDRVYDFAIPGLLLHALEFGKTDSLAKWIEVRPHNAVNVLDTHDGIGVIDIGSDQMDRSLLGLVPDEEVDALVESIHRNSNGESQEATGAAASNLDLYQVNCTYYSALGSDDQKYIAARAVQFFMPGVPQVYYVGALAGKNDMELLKNTNVGRDINRHYYSAAEVAQEVERPVVKALNALGRFRNTLSAFDGEFSYTEADGVLTMTWADDATSAKLTFAPQAGAHDVSVARLEWKDSAGEHATDDLIANPPVVA

SEQ ID NO: 4 - protein, 504 aa, Unknown
  Thermanaerothrix daxensis
MKNQVQLITYVDRLGSGNIKTLHQLLRGPLAGLFGGVHLLPFYYPIKGADAGFDPIDHTRVDPCLGSWEDIRALGQDVDLMADLIVNHISSSSPQFLDYLEKGDDSIYKDLFLTMSSVFPNGATEADLLTIYRPRPGLPFSYITLKNGQKRLLWTTFSRQQIDINVLHPMGREYLHSVLRTLHENGIRMVRLDAVGYAVKKAGTTCFMIPETFDFIENLTHQAQELGMEVLVEIHSHYRKQIEIARQVDRVYDFALPPLVLHAIFNRTAYYLKQWLSISPRNAITVLDTHDGIGVIDIGADSSDPQNYPGLIPPEELEALVEQIHLNSNGQSRLASGAAASNLDLYQVNCTFYDALGRNDRDYLLARAIQFFSPGIPQVYYVGLLAGENDMDLLARTGVGRDINRHYYTLEEIAQAIQRPVVQSLFRLIRFRNQHPAFNGAFSMPESPDSRLILRWDNGAAWAVLEVDFAAGTFSISGSPLEGAEPIEALPGAHPDNRYGGIAT

SEQ ID NO: 5 - DNA, 1518 bp, Bifidobacterium pseudolongum
gtgaagaacaaagtgcagctcatcacctatgcggaccgagtgggcgatggcaatctcgcatccctgaccgacatcctgcgcacccgtttcgccggcgtgtatgaaggcgtgcatatcctgccgttcttcacgccgttcgacggcgccgacgctggcttcgaccccatcgaccacacgaaagtcgacccgcgcctcggtgattgggatgacatcgccgagctctccaagacacacgacatcatggtcgacgcgatcgtcaaccacatgagctggcagtcgcgccagttccaggatgtgctcaagcacggcgaagagtccgagtattacccgatgttcctgacgatgagctcggtcttcccggacggtgcgaccgaagaggagctcgccgggatctaccgcccgcgcccgggcctgccgttcacccactacaccttcgccggcaagacgcgcctggtgtgggtcacgttcacgccgcagcaggtggacatcgacaccgactccgccgaaggctgggcgtacctgatgtcgatcttcgaccggatgggcacatcgcacgtcaagtacattcgcctcgatgccgtcggttatggcgcaaaagaggccggcacgagctgcttcatgacacccaagaccttcgctctgatctcccgtttgcgcgaggagggcgccaagcgcggactcgagatcctcatcgaggtgcactcgtactacaagaagcaggtggagatcgccgcgaaggtggaccgcgtctatgacttcgcgctgcccccgctgctgctgcattcgctgttcaccggccgtgtggacgcgctcgcgcactggaccgagatccgcccgaacaacgccgtcaccgtgctggacacgcacgatggcatcggcgtcatcgacatcggctccgaccagctcgaccgctcgctcaagggcctcgttcccgacgaggacgtcgacgccatggtcgagacgatcgcgaagaacacgcacggcgagtcgaaggctgcgaccggcgccgccgcgtcgaacctcgacctgtaccaggtgaactccacgtattattccgcgctcggcggcaacgaccagcactacatcgctgcgcgcgccgtgcagttcttcctgccgggtgtgccgcaggtgtactacgtcggcgcgctcgccggcagcaacgacatggagttgctcaagcgcaccaatgtcggccgcgacatcaaccgccactactacacgaccgcggagatcgacgcgaacctcgagcggcccgtcgtacgcgcgctcaacgcgctcgcgaagttccgcaacgagctgcccgcgttcgatggcggcttcaactacgccgtcgacggcgagacgatgagtttcacgtggaacgatggtgcgacttccgccaccctgcgcttcacaccttcgcggggcatgggcgcggacaacgcccaacccgtggccgtgctcacgtgggcagacgccgccggcgagcacacgagcgacgacctgattgcgaatccgcctgtggtgcacatggactga

SEQ ID NO: 6 - DNA, 58 bp, Artificial Sequence
  Foward primer
gcggtacctagaactaaacttaaggagacttattatgaagaacaaagtgcagctcatc

SEQ ID NO: 7 - DNA, 28 bp, Artificial Sequence
  reverse primer
gtctagattagtccatgtgcaccacagg

SEQ ID NO: 8 - DNA, 1503 bp, Unknown
  Alloscardovia criceti
atgaagaacaaagttcaattaattacctatgcagatcgccttggtgatgggacattgcagtctatgaccgagaccatccgcaagcattttgatggcgtgtatgagggcgtgcatattctcccattcttcacaccgttcgacggagctgatgcaggcttcgacccagtggatcacacgcaagtggatccacgtttgggctcttgggatgacgtggcagagctttccaagacgcacgacattatggtcgataccattgtgaaccacatgtcgtgggaatccaagcagttccaggacgtgatggctaagggtgaggaatctgagtattatccaatgttcctgaccatgtcttcgattttcccagatggcgtcaccgaagaggatttgaccgccatttatcgtccacgtccaggtctgccatttacgcattacacctggggtggcaagacgcgtctggtctggacaacctttacgcctcagcaggtggatattgataccgactcagaaatgggttggaattatctgctcaccattttggatcagctgtctcagtcgcatgtatcccagatccgtttggatgcggtgggctacggtgcgaaggaaaagaattcgtcctgcttcatgacgccgaagaccttcaagctcatcgagcgcattaaggctgagggcgagaagcgtggcttggaaaccttgattgaggtgcattcctactacaagaagcagatcgaaattgcttccaaggtggatcgcgtgtatgacttcgccatcccgggtctgcttttgcatgctttggaattcggcaagaccgattcgttggccaagtggattgaagtacgtccgcacaatgcggtcaacgtactggatacgcacgatggcattggcgttatcgacatcggctctgaccagatggatcgctccttgctgggtctcgtaccagatgaggaagtcgatgctctggtggagtccattcatcgcaattccaacggcgaatcccaggaagcaaccggtgcggccgcatctaaccttgatttgtatcaggtcaactgcacgtactactccgctttgggtagcgatgaccagaagtacatcgctgcgcgtgccgtgcagttcttcatgccaggcgtgccacaggtatattatgttggcgctttggcgggtaagaatgatatggagctgctcaagaacaccaatgtgggccgcgatattaatcgtcactactactccgcagccgaagtcgctcaggaagtggagcgcccagtggtgaaggctctcaatgcattgggtcgtttccgcaatactctgtccgccttcgatggtgaatttagctacaccgaagcagacggcgtgcttaccatgacttgggcggatgacgctaccagcgccaagctcaccttcgcccctcaggccggtgctcacgatgtatccgtagcccgcttggagtggaaggatagtgctggcgagcatgctaccgatgatctcattgcaaacccaccagtggtggcatag

SEQ ID NO: 9 - DNA, 55 bp, Artificial Sequence
  foward primer
gcggtacccgaagtaaggaggtttagatatgaagaacaaagttcaattaattacc

SEQ ID NO: 10 - DNA, 25 bp, Artificial Sequence
  reverse primer
gtctagattatgccaccactggtgg

SEQ ID NO: 11 - DNA, 1515 bp, Unknown
  Thermanaerothrix daxensis
atgaaaaaccaagttcaactcatcacctacgtagaccgcctgggaagcggtaacatcaaaacactccaccaattgctgcgtggccccctggctggcttattcggcggtgtccaccttctccccttctattaccccattaagggagccgatgccgggtttgatccgattgatcacacccgggttgacccctgtctgggcagttgggaggatatcagggcattggggcaggatgttgacttaatggcggacttaatcgttaaccatatttcatcgtcctcgccccagttcctggattatttggagaagggggacgactcgatctacaaagatttgtttcttacgatgagcagtgttttcccgaacggtgccaccgaagccgacttattgaccatttatcgccccagacccggtttgcctttttcttatataaccctgaagaacggccaaaaacgtttattgtggaccaccttctccaggcagcagattgacatcaatgtattgcaccctatggggagagagtacctgcactcggtattgcgcactctgcatgaaaacggcattcgcatggtgcgtctggatgctgttgggtatgccgtcaaaaaggcgggaaccacttgttttatgatccccgagacgtttgattttattgaaaacctgacccatcaagcccaggaattggggatggaggtcttggtcgaaatccactcgcactatcgcaagcaaattgagattgcccgtcaggtggatcgtgtctacgattttgctttgccccccctggttctgcacgccatattcaatcgcacggcatactacctaaagcaatggctgagtatcagcccgcgcaatgcgattaccgttctggatacgcatgatggcattggggtgattgacatcggcgccgacagcagtgatccacaaaactaccccggtctcattcctccggaagaattagaggctttagtggagcaaattcatcttaacagcaacgggcagagccgtctggccagcggtgccgccgcctccaacttggatttatatcaggtgaattgcactttttatgatgcgctcgggcgcaacgaccgtgattatttgttggcacgcgccattcagttcttctcgccgggcatccctcaggtttactacgtgggtttgctggcgggcgaaaatgacatggatctgctggcccgcacgggtgtcgggcgtgatatcaaccggcattactacaccctggaggagattgcccaggccatccagcgccccgtggtgcaatcgctgttccggctgattcgctttcgcaaccagcaccccgcttttaacggggcgtttagcatgcccgaatccccggattcccggctcatcttgcgttgggataatggggcagcctgggcggtattagaggtggattttgctgccgggaccttttccatttccggttcgccgttagagggggcggaacccatagaggcgttaccaggtgcccacccagacaaccgctacgggggtatcgccacttaa

SEQ ID NO: 12 - DNA, 73 bp, Artificial Sequence
  foward primer
gcggtacccggcgctatcggctttcctttcacaggaggacatttattatgaaaaaccaagttcaactcatcac

SEQ ID NO: 13 - DNA, 23 bp, Artificial Sequence
  reverse primer
gtctagattaagtggcgataccc

SEQ ID NO: 14 - DNA, 1503 bp, Artificial Sequence
  Alloscardovia criceti BamHI and NotI position substitution
atgaagaacaaagttcaattaattacctatgcagatcgccttggtgatgggacattgcagtctatgaccgagaccatccgcaagcattttgatggcgtgtatgagggcgtgcatattctcccattcttcacaccgttcgacggagctgatgcaggcttcgacccagtggatcacacgcaagtcgatccacgtttgggctcttgggatgacgtggcagagctttccaagacgcacgacattatggtcgataccattgtgaaccacatgtcgtgggaatccaagcagttccaggacgtgatggctaagggtgaggaatctgagtattatccaatgttcctgaccatgtcttcgattttcccagatggcgtcaccgaagaggatttgaccgccatttatcgtccacgtccaggtctgccatttacgcattacacctggggtggcaagacgcgtctggtctggacaacctttacgcctcagcaggtggatattgataccgactcagaaatgggttggaattatctgctcaccattttggatcagctgtctcagtcgcatgtatcccagatccgtttggatgcggtgggctacggtgcgaaggaaaagaattcgtcctgcttcatgacgccgaagaccttcaagctcatcgagcgcattaaggctgagggcgagaagcgtggcttggaaaccttgattgaggtgcattcctactacaagaagcagatcgaaattgcttccaaggtggatcgcgtgtatgacttcgccatcccgggtctgcttttgcatgctttggaattcggcaagaccgattcgttggccaagtggattgaagtacgtccgcacaatgcggtcaacgtactggatacgcacgatggcattggcgttatcgacatcggctctgaccagatggatcgctccttgctgggtctcgtaccagatgaggaagtcgatgctctggtggagtccattcatcgcaattccaacggcgaatcccaggaagcaaccggtgctgccgcatctaaccttgatttgtatcaggtcaactgcacgtactactccgctttgggtagcgatgaccagaagtacatcgctgcgcgtgccgtgcagttcttcatgccaggcgtgccacaggtatattatgttggcgctttggcgggtaagaatgatatggagctgctcaagaacaccaatgtgggccgcgatattaatcgtcactactactccgcagccgaagtcgctcaggaagtggagcgcccagtggtgaaggctctcaatgcattgggtcgtttccgcaatactctgtccgccttcgatggtgaatttagctacaccgaagcagacggcgtgcttaccatgacttgggcggatgacgctaccagcgccaagctcaccttcgcccctcaggccggtgctcacgatgtatccgtagcccgcttggagtggaaggatagtgctggcgagcatgctaccgatgatctcattgcaaacccaccagtggtggcatag

SEQ ID NO: 15 - DNA, 36 bp, Artificial Sequence
  foward primer
gctggatccatgaagaacaaagttcaattaattacc

SEQ ID NO: 16 - DNA, 28 bp, Artificial Sequence
  reverse primer
ctgcggccgcttatgccaccactggtgg

SEQ ID NO: 17 - DNA, 33 bp, Artificial Sequence
  foward primer
gctggatccatgaagaacaaagtgcagctcatc

SEQ ID NO: 18 - DNA, 31 bp, Artificial Sequence
  reverse primer
ctgcggccgcttagtccatgtgcaccacagg

SEQ ID NO: 19 - DNA, 35 bp, Artificial Sequence
  foward primer
gctggatccatgaaaaaccaagttcaactcatcac

SEQ ID NO: 20 - DNA, 26 bp, Artificial Sequence
  reverse primer
ctgcggccgcttaagtggcgataccc

SEQ ID NO: 21 - protein, 289 aa, Unknown
  Clostridium hylemonae
MKHGIYYAYWEQEWAADYKRYVEKVAKLGFDILEIGAGPLPEYAEQDVKELKKCAQDNGITLTAGYGPTFNHNIGSSDAGVREEALEWYKRLFEVLAELDIHLIGGALYSYWPVDFANADKTEDWKWSVEGMQRLAPAAAKYDINLGMEVLNRFESHILNTAEEGVKFVEEVGMDNVKVMLDTFHMNIEEQSIGGAIRRAGKLLGHFHTGECNRMVPGKGRIPWREIGDALRDIGYDGTAVMEPFVRMGGQVGADIKVWRDISRGADEAQLDDDARRALEFQRYMLEWK

SEQ ID NO: 22 - protein, 336 aa, Lactobacillus reuteri
MKALVLTGKKQLEIEDIKEPEIKPDEVLIHTAYAGICGTDKALYAGLPGSASAVPPIVLGHENSGVVTKVGSEVTNVKPGDRVTVDPNIYCGQCKYCRTQRPELCEHLDAVGVTRNGGFEEYFTAPAKVVYPIPDDVSLKAAAVVEPISCAMHGVDLLETHPYQKALVLGDGFEGQLFAQILKARGIHEVTLAGRSDEKLENNRKHFGVKTINTTKEEIPADAYDIVVEAVGLPATQEQALAAAARGAQVLMFGVGNPDDKFSVNTYDVFQKQLTIQGAFINPYTFEDSIALLSSGVVDPLPLFSHELDLDGVEGFVSGKLGKVSKAVVKVGGEEA

SEQ ID NO: 23 - protein, 401 aa, Mycobacterium vaccae
MAKVLCVLYDDPVDGYPKTYARDDLPKIDHYPGGQILPTPKAIDFTPGQLLGSVSGELGLREYLESNGHTLVVTSDKDGPDSVFERELVDADVVISQPFWPAYLTPERIAKAKNLKLALTAGIGSDHVDLQSAIDRNVTVAEVTYCNSISVAEHVVMMILSLVRNYLPSHEWARKGGWNIADCVSHAYDLEAMHVGTVAAGRIGLAVLRRLAPFDVHLHYTDRHRLPESVEKELNLTWHATREDMYPVCDVVTLNCPLHPETEHMINDETLKLFKRGAYIVNTARGKLCDRDAVARALESGRLAGYAGDVWFPQPAPKDHPWRTMPYNGMTPHISGTTLTAQARYAAGTREILECFFEGRPIRDEYLIVQGGALAGTGAHSYSKGNATGGSEEAAKFKKAV